(19) United States
(12) Patent Application Publication
(10) Pub. No.: US 2026/0048168 A1
(43) Pub. Date: Feb. 19, 2026

(54) WOUND TREATMENT SYSTEM

(71) Applicant: Kimmerling Holdings Group, LLC, Marietta, GA (US)

(72) Inventors: Kirk A. Kimmerling, Kennesaw, GA (US); Marc D. Etchells, Marietta, GA (US)

(21) Appl. No.: 19/076,360

(22) Filed: Mar. 11, 2025

Related U.S. Application Data
(60) Provisional application No. 63/564,160, filed on Mar. 12, 2024.

(51) Int. Cl. A61L 15/44; A61F 13/00; A61F 13/511; A61L 15/42

(52) U.S. Cl. CPC A61L 15/44; A61F 13/00063; A61F 13/51121; A61L 15/425; A61F 2013/5113; A61L 2300/208; A61L 2300/404

(57) ABSTRACT

Disclosed herein a wound care systems including an active layer and a foam layer. The wound care system can include one or more active agents, for example a QAS compound. In certain aspects, the wound care system includes K21.

### CROSS-REFERENCE TO RELATED APPLICATION

This application claims the benefit of U.S. Provisional Application 63/564,160, filed Mar. 12, 2024, the contents of which are hereby incorporated in its entirety.

### FIELD OF THE DISCLOSURE

The disclosure relates to wound treatment systems. The systems include a detachable, resorbable layer and a hydrophilic foam.

### BACKGROUND

Bandage and wound dressing systems are known in the art. Bandages are applied to cover and protect open wounds. Bandages typically include a wound covering portion and a base substrate to which the wound covering portion is affixed.

There remains a need for improved bandage and wound dressing systems. There remains a need for improved bandage and wound dressing systems which promote healing processes. There remains a need for improved bandage and wound dressing systems that treat and prevent bacterial infections in and around wounds. There remains a need for improved compositions and methods for the treatment of wounds.

### BRIEF DESCRIPTION OF THE FIGURES

FIG. 1 depicts a side view of a 4×4 dressing.

FIG. 2 depicts a side view of a 4×4 dressing with a saturation indicator.

FIG. 3 depicts application of the active layer with foam cast on top.

FIG. 4 depicts application of the active layer directly onto a nonwoven, knit, woven, film, or foam surface.

FIG. 5 depicts the chemical structure of K21.

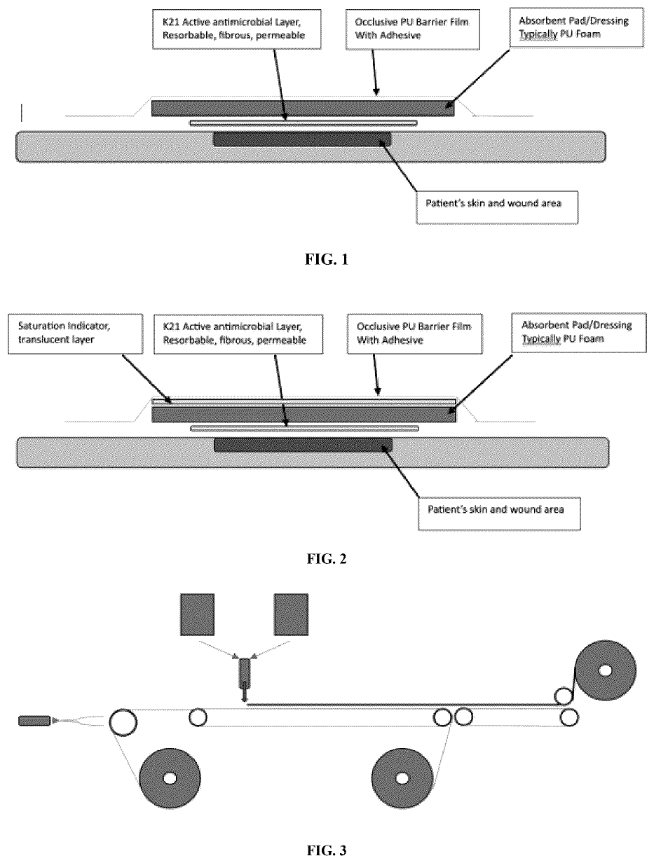

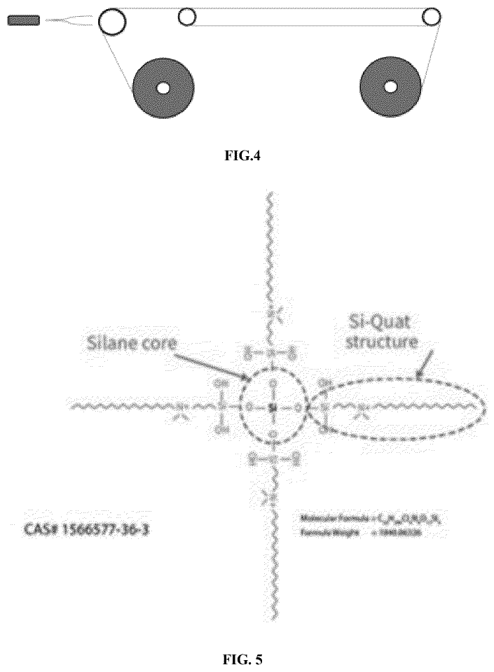

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes, from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Compounds disclosed herein may be provided in the form of acceptable salts. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate.

The term “alkyl” refers to a radical of a straight-chain or branched hydrocarbon group having a specified range of carbon atoms (e.g., a “C₁₋₁₆ alkyl” can have from 1 to 16 carbon atoms). An alkyl group can be saturated or unsaturated, i.e., an alkenyl or alkynyl group. Unless specified to the contrary, an “alkyl” group includes both saturated alkyl groups and unsaturated alkyl groups.

When a range of values is listed, it is intended to encompass each value and sub-range within the range. For example, “C₁₋₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “alkoxy” refers to an alkyl group, as defined herein, appended to the parent molecular moiety through an oxygen atom.

The term “aryl” refers to a radical of a monocyclic or polycyclic (e.g., bicyclic or tricyclic) 4n+2 aromatic ring system (e.g., having 6, 10, or 14 T electrons shared in a cyclic array) having 6-14 ring carbon atoms and zero heteroatoms provided in the aromatic ring system (“C₆₋₁₄ aryl”). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents.

“Aralkyl” is a subset of “alkyl” and refers to an alkyl group substituted by an aryl group, wherein the point of attachment is on the alkyl moiety.

Disclosed herein are wound care systems including one or more layers having an active agent. In certain implementations, the wound care system includes at least one antimicrobial agent as the active agent. In some implementations, the wound care system includes a QAS, for example K21.

In some implementations, the wound care system includes an active layer, which can include an active agent such as an antimicrobial agent, for instance a QAS, especially K21. The active layer may include a single antimicrobial agent, or may contain multiple antimicrobial agents, for example two agent, three agents, etc. In certain implementations the active layer is a lubricious, resorbable, detachable, permeable, and fibrous layer.

An overall protective, absorbent, and occlusive pad, wrap or dressing which may include skin adhesive or self-cohesive properties and may also be resorbable, the pad may also include an antimicrobial agent, the same or different than the antimicrobial agent in the active layer. The antimicrobial agent in the pad can be a QAS, especially K21. The pad may include a single antimicrobial agent, or may contain multiple antimicrobial agents, for example two agent, three agents, etc.

In certain implementations the wound care system includes a release liner disposed against the adhesive. When removed, it exposes the adhesive for use. The release liner is generally paper or film with a silicone layer on one or both surfaces allowing the adhesive of the dressing to be peeled away without damage. This release liner can also be a polycarbonate, polyethylene, polypropylene, or polyester film.

The wound care system can also include a spray applied liquid that provides rapid disinfectant/cleanser and anti-inflammatory benefits which may optionally include a pain relief composition. The spray may include an antimicrobial agent, the same or different than the antimicrobial agent in the active layer. The antimicrobial agent in the pad can be a QAS, especially K21. The spray may include a single antimicrobial agent, or may contain multiple antimicrobial agents, for example two agent, three agents, etc. The spray may include water, ethanol, or a combination thereof. Thickening agents may be utilized to promote adhesion such as hydrogels, cellulose, starch, gums, etc. Surfactants may be utilized to promote deep penetration for optimized cleaning and/or antimicrobial activity. The spray can include an active agent, for instance an antimicrobial agent, an analgesic agent, an anti-inflammatory or a combination thereof. In certain implementations the spray can include lidocaine, capsaicin, menthol, or a combination thereof, optionally further including a QAS as described herein.

The system may also include a saturation indicator to alert when the absorbent bandage is nearing full capacity and should be changed. The saturation indicator may be disposed between the foam layer and the occlusive barrier film. The saturation indicator can be white non-woven or fabric that is opaque when dry and becomes visibly transparent when saturated, thereby permitting visualization of the foam layer, which may be pigmented.

The wound care system can further include an adhesive, for example a gentle release formulation such as a silicone. For systems in which a stronger adhesive is desired acrylic adhesives, urethane adhesives, and or hydrocolloid adhesives may be included. In certain implementations a QAS, for example K21, may be mixed as an ethanol solution with an acrylic solvent cast adhesive without compromising either the adhesive properties of the acrylic or the antimicrobial properties of the QAS. In certain implementations a QAS, for example K21, may be mixed as an ethanol solution with either or both phases of a two-part addition cure silicone adhesive without compromising either the adhesive properties of the silicone or the antimicrobial properties of the QAS.

In some implementations the active layer is a resorbable fibrous layer, which may be obtained via electrospinning. The active layer can include small diameter fibers which provide controlled release of the active agents disposed therein. The fibrous layer can be breathable and non-occlusive, measured with ASTM D737-18 test method with a minimum permeability rate of 1.500 cubic ft/minute/square ft at 0.5″ air pressure. The fibrous layer can be bioresorbable and forms a protective layer when applied to a wound. The protective layer can reduce direct oxygen contact with the wound thereby reducing pain.

The active layer can be a detachable layer that effectively separates from the wound care system when it contacts a wound. Thus, other layers of the wound care system can be removed while the active layer remains in contact with the wound. The active, detachable layer promotes release of the active agent. In some implementations the active, detachable layer is clear to permit visual inspection of the wound.

An electrospun active layer can be prepared by electrospinning a solution containing the active agent such as antimicrobial agent, e.g. a QAS preferably K21 and one or more bioresorbable polymers.

In certain implementations, the bioresorbable polymer can include one or more of saccharides, surfactants (e.g., poloxamers, etc.), collagen or gelatin, alginate, cellulose-based or carboxymethyl cellulose (CMC), polyvinyl alcohol (PVA or PVOH), polyethylene oxide (PEO), polylactic acid (PLA), glycolic acid, polycaprolactone (PCL), a copolymer thereof, or a combination thereof. In some implementations the bioresorbable polymer can include PLGA. In some implementations the bioresorbable polymer can include a poly(lactic acid) polymer, such as poly(L-lactic acid), poly(D-lactic acid), poly(D/L-lactic acid). In certain implementations the bioresorbable polymer can include PVA, PEO, CMC, or a combination thereof.

In certain implementations the active layer can include fibers such as core/sheath, side-by-side, or island-in-the-sea structures. In such implementations, the active agent, for example K21 may be in both fiber phases, or in a single phase. For example, the active agent may be present in the core fiber, but not the sheath. The active agent may be present in the island fiber, but not the sea.

The fibers in the active layer can have an average fiber diameter from 10-1,000 nm, from 10-100 nm, from 10-50 nm, from 50-100 nm, from 50-250 nm, from 75-500 nm, from 100-250 nm, from 250-500 nm, or from 500-1,000 nm. In certain implementations the basis weight of the active layer can be from 0.5-200 gsm, from 0.5-25 gsm, from 5-25 gsm, from 25-50 gsm, from 50-100 gsm, from 50-150 gsm, or from 100-200 gsm. In certain implementations, the basis weight is from 10-50 gsm.

The weight ratio of the bioerodible polymer to active agent can be from 5:1 to 1:5, 2.5:1 to 1:2.5, 2.5:1 to 1:1, 2:1 to 1:1, or 1.5:1 to 1:1. The concentration of bioerodible polymer in the solution can be from 0.01-1,000 mg/ml, from 0.1-1,000 mg/ml, 1-1,000 mg/ml, from 0.1-500 mg/ml, from 0.1-250 mg/ml, from 0.1-100 mg/ml, from 0.1-50 mg/ml, from 1-100 mg/ml, from 1-50 mg/ml, from 5-50 mg/ml, from 10-50 mg/ml, or from 15-25 mg/ml.

In certain implementations, the total solids content in the electrospinning solution can be from 0.5-20 wt. %, from 0.5-5 wt. %, from 1-3 wt. %, from 1-5 wt. %, from 2.5-7.5 wt. %, or from 5-10 wt. %.

In certain implementations the active agent can be in the electrospinning solution at a concentration from 0.1-10 wt. %, from 0.1-1 wt. %, from 0.5-1 wt. %, from 0.5-2.5 wt. %, from 1-2.5 wt. %, or from 2.5-5 wt. %. In certain implementations, the electrospinning solution can include a QAS, for example K21, at a concentration from 0.1-10 wt. %, from 0.1-1 wt. %, from 0.5-1 wt. %, from 0.5-2.5 wt. %, from 1-2.5 wt. %, or from 2.5-5 wt. %.

In certain implementations, the active layer can include more than one layer of electrospun fibers, differing by one or more of fiber diameter, fiber basis weight, polymer composition, active agent composition (different active agents, different concentrations of active agents, or both). For example, an active layer can include a proximal active layer, which contacts the wound, and a distal active layer, which is disposed between the proximal active layer and the foam layer. In certain implementations, the proximal active layer can include a greater amount of active agent relative to the distal active layer, for example a greater amount of K21. In certain implementations, the proximal active layer can include one or more analgesics. In certain implementations, the distal active layer can include one of more analgesics and/or anti-inflammatory agents.

In certain implementations the active layer can be deposited on a drum and later incorporated into the wound care system. In some implementations the active layer can be directly deposited on the foam layer, on the release liner, or on different layer of the wound care system. In certain implementations, the fibers are randomly oriented.

In some implementations, the active layer can be prepared using gel coating via an extruder, 3D printing including BioPrinting, roll coating or printing including UV-curing for crosslinking, spray or atomization as a liquid, as a foam or foamed layer, as an aerated gel or foam which is then dried or cured, or via a die cutting or robotic assembly process where the degradable K21 layer is cut to size and placed where required. If BioPrinting is utilized, live cells or organisms may be imbedded into or coated onto the fibers/filaments/structure.

The active layer can be cast or extruded as a film, and then laminated to the foam layer. The film may be perforated for fluid and/or gaseous breathability.

In some implementations the active layer can be applied as a continuous layer covering the entire surface of the foam layer. In some implementations the active layer can be disposed on only a portion of the foam, typically disposed in the center.

The wound care system can also include an absorbent foam layer. The foam layer can be composed of a polyurethane. The foam layer can provide the surface for the placement of an electrospun fibrous scaffold which contains one or more active layers, for example K21 as an antimicrobial. The foam is highly absorbent with a typical thickness of <1 mm to >5 mm and a minimum fluid-holding capacity of 10× gram/gram for the capture of wound drainage or exudate (serous, sanguineous, serosanguineous, and purulent).

In some implementations the foam layer can include an active agent such as a antimicrobial agent, for instance a QAS, especially K21. The foam layer may include a single antimicrobial agent, or may contain multiple antimicrobial agents, for example two agent, three agents, etc. In certain implementations the foam layer can include a particulate QAS, as defined herein.

In some implementations the active agent, for example an antimicrobial agent such as a QAS, preferably K21, can be dissolved or dispersed in one or both of the precursor components of a two-part polyurethane. In some implementations the active agent can be present in the polyol component. In some implementations the active agent can be present in the water/surfactant component. In some implementations the active agent, for example an antimicrobial agent, for example a QAS like K21 is present in the polyol component at a concentration from 0.01-5 wt. %, from 0.01-0.1 wt. % from 0.1-0.5 wt. %, from 0.1-1 wt. %, from 0.5-1 wt. %, from 1-2.5 wt. %, from 1-5 wt. %, or from 2.5-5 wt. %. In some implementations the active agent, for example an antimicrobial agent, for example a QAS like K21 is present in the water/surfactant component at a concentration from 0.01-5 wt. %, from 0.01-0.1 wt. %, from 0.01-5 wt. %, from 0.1-0.5 wt. %, from 0.1-1 wt. %, from 0.5-1 wt. %, from 1-2.5 wt. %, from 1-5 wt. %, or from 2.5-5 wt. %.

In certain implementations the foam layer can be surface treated with the active agent, to provide a foam layer with a surface coating of agent, without active agent dispersed throughout the foam. In certain implementations the foam can be dip coated with the active agent, for example an antimicrobial agent, for example a QAS like K21. The foam may be submerged in, sprayed with, or painted with a solution including the active agent.

The foam layer can also include one or more surfactants to promote fluid transport and provide lubricity to the foam layer, thereby reducing pain when the foam layer is removed from a wound. The foam layer can be pigmented on one or both sides.

In certain implementations the foam layer can include an occlusive barrier film disposed on the side of the layer distal to the active layer. The occlusive barrier film prevents pathogen ingress into the system and prevents fluid leakage out of the wound care system.

The wound care systems disclosed herein can be used to treat a variety of different wounds. In certain implementations, the wound care system can be used to treat trauma wounds, surgical wounds, burn wounds, or ulcer wounds.

Also disclosed herein are kits including the dressing of any preceding claim, and a liquid spray composition including a spray antimicrobial agent.

The liquid spray composition can include QAS, preferably K21, optionally in combination with at least one additional liquid spray therapeutic agent. The QAS can be present in the spray at a concentration of 0.1-5 wt. %, from 0.1-1 wt. %, from 0.5-2.5 wt. %, from 1-2.5 wt. %, from 1-5 wt. %, or from 2.5-5 wt. %. Suitable additional liquid spray therapeutic agent comprises an antimicrobial agent, an analgesic, an anti-inflammatory, a cannabinoid, a growth factor, or a combination thereof. The spray composition may include water, ethanol or a combination thereof. In certain implementations the spray composition includes one or more additional therapeutic agents as disclosed herein. The spray composition may be disposed in a suitable container capable of dispensing the composition (for instance using a hand pump) to a wound. Subsequent to application of the spray, the wound care systems disclosed herein may be applied to the wound.

In some implementations, the wound care system includes a quaternary ammonium siloxane (“QAS”) compound. In some implementations, the QAS compound is a compound of Formula 1:

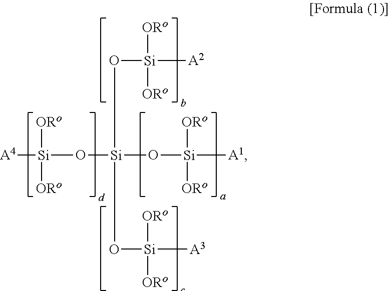

wherein:
a is 0, 1, 2, 3, 4, 5, 6, 7, 8, 9, or 10;
b is 0, 1, 2, 3, 4, 5, 6, 7, 8, 9, or 10;
c is 0, 1, 2, 3, 4, 5, 6, 7, 8, 9, or 10;
d is 0, 1, 2, 3, 4, 5, 6, 7, 8, 9, or 10;
A¹ has the formula —(CH₂)ₑ—R^{a};
wherein e is 0, 1, 2, 3, 4, 5, 6, 7, 8, 9, or 10, preferably 2, 3, or 4, most preferably 3
R^{a} is selected from H, OR^{o}, or a group having the formula:

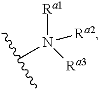

wherein R^{a1}, R^{a2}, and R^{a3} are each independently selected from C₁₋₂₅alkyl, wherein any two or more of R^{a1}, R^{a2}, and R^{a3} may together form a ring;
A² has the formula —(CH₂)_{f}—R^{b};
wherein f is 0, 1, 2, 3, 4, 5, 6, 7, 8, 9, or 10, preferably 2, 3, or 4, most preferably 3;
R^{b} is selected from H, OR^{o}, or a group having the formula:

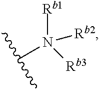

wherein R^{b1}, R^{b2}, and R^{b3} are each independently selected from C₁₋₂₅alkyl, wherein any two or more of R^{b1}, R^{b2}, and R^{b3} may together form a ring;
A³ has the formula —(CH₂) g-R^{c};
wherein g is 0, 1, 2, 3, 4, 5, 6, 7, 8, 9, or 10, preferably 2, 3, or 4, most preferably 3;
R^{c} is selected from H, OR^{o}, or a group having the formula:

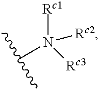

wherein R^{c1}, R^{c2}, and R^{c3} are each independently selected from C₁₋₂₅alkyl, wherein any two or more of R^{c1}, R^{c2}, and R^{c3} may together form a ring;
A⁴ has the formula —(CH₂)ₕ—R^{d};
wherein h is 0, 1, 2, 3, 4, 5, 6, 7, 8, 9, or 10, preferably 2, 3, or 4, most preferably 3;
R^{d} is selected from H, OR^{o}, or a group having the formula:

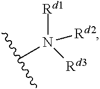

wherein R^{d1}, R^{d2}, and R^{d3} are each independently selected from C₁₋₂₅alkyl, wherein any two or more of R^{d1}, R^{d2}, and R^{d3} may together form a ring; and
R^{o} is in each case independently selected from H, CH₃, CH₂CH₃, or CH(CH₃)₂.

In certain embodiments, each of a, b, and c is 0, and each of A¹, A², and A³ is OR^{o}. In such embodiments, A⁴ has the formula —(CH₂)ₕ—R^{d} wherein R^{d} has the formula:

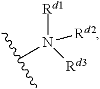

In other embodiments, each of a, b, c, and d are 1, R^{o} is each case is H, e, f, g, and h are each 1, 2, 3, or 4; and each of R^{a}, R^{b}, R^{c}, and R^{d} have the formula:

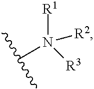

wherein R¹, R², and R³ are each independently selected from C₁₋₂₅alkyl, wherein any two or more of R¹, R², and R³ may together form a ring. Such embodiments are compound of Formula (Ia):

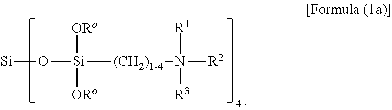

The skilled person understands that each quaternary nitrogen formally bears a cationic charge (not depicted), and as such will be balanced by a suitable number or kind of anionic species, as defined herein.

In certain embodiments, R¹ and R² are each C₁₋₃alkyl, or R¹ and R² together with the nitrogen atom form a 5-7 membered ring, and R³ is C₃₋₂₅alkyl, preferably C₆₋₂₅alkyl, more preferably, C₁₀₋₂₅alkyl, and even more preferably C₁₄₋₂₂alkyl. In some preferred embodiments, R¹ and R² are each methyl.

In certain embodiments, the QAS can be in particulate form, for instance as a particle including a portion of groups having the formula —(CH₂)ₚ—R^{p} covalently bonded to a silica particle, wherein p is 0, 1, 2, 3, 4, 5, 6, 7, 8, 9, or 10, preferably 2, 3, or 4, most preferably 3, and R^{p} has the formula:

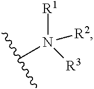

wherein R¹, R², and R³ are each independently selected from C₁₋₂₅alkyl, wherein any two or more of R¹, R², and R³ may together form a ring. In certain embodiments, R¹ and R² are each C₁₋₃alkyl, or R¹ and R² together with the nitrogen atom form a 5-7 membered ring, and R³ is C₃₋₂₅alkyl, preferably C₆₋₂₅alkyl, more preferably, C₁₀₋₂₅alkyl, and even more preferably C₁₄₋₂₂alkyl. In some preferred embodiments, R¹ and R² are each methyl.

In some embodiments, the groups having the formula —(CH₂)ₚ—R^{p} will be more or less homogenously distributed throughout the particle. In other embodiments, the portion of groups having the formula —(CH₂)ₚ—R^{p} will be largely contained on the outer surface of the particle. For example, the particle can be a core-shell particle containing an interior core of SiO₂, and an exterior core of silicon atoms covalently bonded to groups having the formula —(CH₂)ₚ—R^{P}. In some embodiments, the particle has the formula:

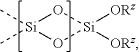

wherein the bracketed portion reflects an indeterminate number of siloxane units forming a core, and R^{z} is independently chosen from H, CH₃, CH₂CH₃, and groups having the formula —(CH₂)ₚ—R^{p}. The skilled person will understand that such a particle may include portions of incompletely bonded silicon atoms in the core, the above depiction is not intended to exclude such possibilities from the invention.

In certain embodiments, the QAS can be a compound (or mixture of compounds) prepared by reacting a compound having the formula Si(OR^{o1})₄ with a compound of Formula (2):

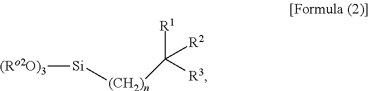

wherein R^{o1} is H, CH₃, CH₂CH₃, or CH(CH₃)₂, and is preferably CH₂CH₃, R^{o2} is H, CH₃, CH₂CH₃, or CH(CH₃)₂, and is preferably CH₃, n is 0, 1, 2, 3, 4, 5, or 6, preferably 2, 3, or 4, most preferably 3.

The molar ratio of compound having the formula Si(OR^{o1})₄ to the compound of Formula (2) can be from 100:1 to 50:1, from 75:1 to 25:1, from 50:1 to 25:1, from 40:1 to 20:1 from 25:1 to 10:1, from 10:1 to 1:10, from 5:1 to 1:5, from 2.5:1 to 1:2.5, from 1.5:1 to 1:1.5, from 10:1 to 5:1, from 7.5:1 to 2.5:1, from 2.5:1 to 1:1, from 1:1 to 1:2.5, from 1:2.5 to 1:7.5, from 1:5 to 1:10, from 1:10 to 1:20, from 1:15 to 1:25, from 1:20 to 1:40, from 1:25 to 1:50, from 1:25 to 1:75, or from 1:50 to 1:100.

In certain embodiments, the compound having the formula Si(OR^{o1})₄ can be combined with the compound of Formula (2) in the presence of an acid reagent or base reagent. The acid reagent can be a strong acid (pKₐ in water <1), for example HCl, HBr, HI, sulfuric acid, phosphoric acid, nitric acid, and the like. The acid reagent can be a weak acid (pKₐ in water >1), for example a carboxylic acid like acetic acid, propionic acid, benzoic acid, and the like. The base reagent can be KOH, NaOH, sodium methoxide, sodium ethoxide, triethylamine, diisopropylethylamine and the like. In certain embodiments, the compound having the formula Si(OR^{o1})₄ can be combined with the compound of Formula (2) in a mixture having a pH less than about 1, greater than about 12, or from about 7-12, from about 8-12, from about 8-11, from about 8-10, from about 9-10, from about 9-12, from about 9-11, from about 9-10, from about 1-7, from about 1-5, from about 1-4, from about 1-3, from about 1-2, from about 2-5, from about 2-4, from about 3-5, or from about 4-6. When the compound having the formula Si(OR^{o1})₄ can be combined with the compound of Formula (2) in an acidic aqueous mixture, or a basic aqueous mixture, the amount water can be present in an amount (relative to the total wt. of the mixture) from about 5-100 wt. %, from about 1-20 wt. %, from about 5-25 wt. %, from about 10-30 wt. %, from about 10-40 wt. %, from about 10-50 wt. %, from about 25-50 wt. %, from about 25-75 wt. %, from about 50-100 wt. %, from about 100-250 wt. %, from about 100-500 wt. %, or greater than 500 wt. %.

In some embodiment, a mixture including the compound having the formula Si(OR^{o1})₄ and compound of Formula (2) can be heated to form the QAS. For example, the mixture can be heated to a temperature between 25-100° C., between 25-100° C., between 25-75° C., between 50-75° C., between 50-100° C., between 75-125° C., between 100-150° C., or to a temperature greater than 150° C.

In some embodiments, the compound having the formula Si(OR^{o1})₄ and compound of Formula (2) can be combined in the absence of a solvent (except for incidental solvent used to deliver an acid or base reagent, e.g., 1 M HCl solution). In other embodiments, the compound having the formula Si(OR^{o1})₄ and compound of Formula (2) can be combined with a solvent, for example a polar protic solvent such as water and lower alcohols such as methanol, ethanol, or propanol; a polar aprotic solvent like acetone, ethyl acetate, DMSO, DMF, THF, or methylene chloride. The solvent can be present in an amount to give a mixture having a concentration (wt/v) of about 0.01-0.1%, 0.1-1%, 0.1-5%, 1-20%, 5-25%, 5-50%, 25-50%, or 25-75%.

In certain embodiments, the compound having the formula Si(OR^{o1})₄ can be combined with one or more compounds having the formula:

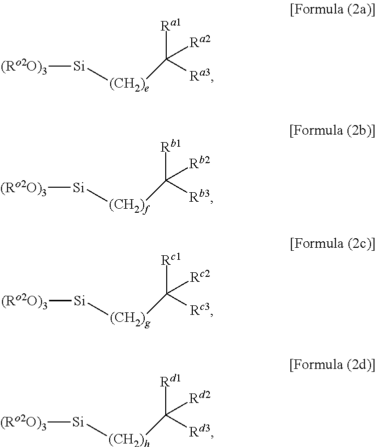

wherein
e is 0, 1, 2, 3, 4, 5, 6, 7, 8, 9, or 10, preferably 2, 3, or 4, most preferably 3;
f is 0, 1, 2, 3, 4, 5, 6, 7, 8, 9, or 10, preferably 2, 3, or 4, most preferably 3;
g is 0, 1, 2, 3, 4, 5, 6, 7, 8, 9, or 10, preferably 2, 3, or 4, most preferably 3;
h is 0, 1, 2, 3, 4, 5, 6, 7, 8, 9, or 10, preferably 2, 3, or 4, most preferably 3;
wherein
R^{a1}, R^{a2}, and R^{a3} are each independently selected from C₁₋₂₅alkyl, wherein any two or more of R^{a1}, R^{a2}, and R^{a3} may together form a ring;
R^{b1}, R^{b2}, and R^{b3} are each independently selected from C₁₋₂₅alkyl, wherein any two or more of R^{b1}, R^{b2}, and R^{b3} may together form a ring;
R^{c1}, R^{c2}, and R^{c3} are each independently selected from C₁₋₂₅alkyl, wherein any two or more of R^{c1}, R^{c2}, and R^{c3} may together form a ring;
R^{d1}, R^{d2}, and R^{d3} are each independently selected from C₁₋₂₅alkyl, wherein any two or more of R^{d1}, R^{d2}, and R^{d3} may together form a ring.

In certain embodiments, R^{a1} and R^{a2} are each methyl, and R^{a3} is a C₁₄₋₂₂alkyl group; R^{b1} and R^{b2} together with the nitrogen atom form a 5-7 membered ring, and R^{b3} is a C₁₄₋₂₂alkyl group; R^{c1}, R^{c2}, and R^{c3} are each independently a C₁₋₄alkyl group, for example methyl, ethyl, or propyl, for example R^{c1}, R^{c2}, and R^{c3} are each methyl, or R^{c1} and R^{c2} are each methyl and R^{c3} is isopropyl, and R^{d1}, R^{d2}, and R^{d3} are each independently a C₃₋₁₁alkyl group, or R^{d1} and R^{d2} are each methyl and R^{d} is a C₅₋₁₅alkyl group.

In other embodiments, the compound of having the formula Si(OR^{o1})₄ is combined with one or more compounds of Formulas (2a), (2b), (2c) and (2d). The molar ratio of compound having the formula Si(OR^{o1})₄ to total amount of the compounds of Formulas (2a), (2b), (2c) and (2d) can be from 100:1 to 50:1, from 75:1 to 25:1, from 50:1 to 25:1, from 40:1 to 20:1 from 25:1 to 10:1, from 10:1 to 1:10, from 5:1 to 1:5, from 2.5:1 to 1:2.5, from 1.5:1 to 1:1.5, from 10:1 to 5:1, from 7.5:1 to 2.5:1, from 2.5:1 to 1:1, from 1:1 to 1:2.5, from 1:2.5 to 1:7.5, from 1:5 to 1:10, from 1:10 to 1:20, from 1:15 to 1:25, from 1:20 to 1:40, from 1:25 to 1:50, from 1:25 to 1:75, or from 1:50 to 1:100. The individual compounds of Formulas (2a), (2b), (2c) and (2d) can be combined as follows (molar ratios):

| Formula (2a) | Formula (2b) | Formula (2c) | Formula (2d) |
|---|---|---|---|
| 100 | 0 | 0 | 0 |
| 0 | 100 | 0 | 0 |
| 0 | 0 | 100 | 0 |
| 0 | 0 | 0 | 100 |
| 25-75 | 25-75 | 0-25 | 0-25 |
| 50 | 50 | 0 | 0 |
| 25-75 | 0-25 | 25-75 | 0-25 |
| 50 | 0 | 50 | 0 |
| 25-75 | 0-25 | 0-25 | 25-75 |
| 50 | 0 | 0 | 50 |
| 0-25 | 25-75 | 25-75 | 0-25 |
| 0 | 50 | 50 | 0 |
| 0-25 | 25-75 | 0-25 | 25-75 |
| 0 | 50 | 0 | 50 |
| 0-25 | 0-25 | 25-75 | 25-75 |
| 0 | 0 | 50 | 50 |
| 25-50 | 25-50 | 25-50 | 0-25 |
| 33.3 | 33.3 | 33.3 | 0 |
| 25-50 | 25-50 | 0-25 | 25-50 |
| 33.3 | 33.3 | 0 | 33.3 |
| 25-50 | 0-25 | 25-50 | 25-50 |
| 33.3 | 0 | 33.3 | 33.3 |
| 0-25 | 25-50 | 25-50 | 25-50 |
| 0 | 33.3 | 33.3 | 33.3 |
| 10-70 | 10-50 | 10-50 | 10-50 |
| 25 | 25 | 25 | 25 |
| 70 | 10 | 10 | 10 |
| 10-50 | 10-70 | 10-50 | 10-50 |
| 10 | 70 | 10 | 10 |
| 10-50 | 10-50 | 10-70 | 10-50 |
| 10 | 10 | 70 | 10 |
| 10-50 | 10-50 | 10-50 | 10-70 |
| 10 | 10 | 10 | 70 |
| 50-85 | 5-15 | 5-15 | 5-15 |
| 5-15 | 50-85 | 5-15 | 5-15 |
| 5-15 | 5-15 | 50-85 | 5-15 |
| 5-15 | 5-15 | 5-15 | 50-85 |
| 90-97 | 1-5 | 1-5 | 1-5 |
| 1-5 | 90-97 | 1-5 | 1-5 |
| 1-5 | 1-5 | 90-97 | 1-5 |
| 1-5 | 1-5 | 1-5 | 90-97 |

In certain implementations, the QAS includes K21, which has the formula:

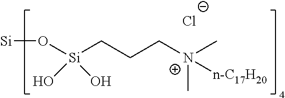

When the wound care system includes a particulate component, the particle can be substantially spherical, or can be non-spherical with an average aspect ratio greater than 1:1.25, greater than 1:1.5, greater than 1:1.75, greater than 1:2.0, greater than 1:2.25, or greater than 1:2.5.

The particles can have a BET surface area of at least 100 m²/g, at least 200 m²/g, at least 300 m²/g, at least 400 m²/g, at least 500 m²/g, at least 750 m²/g, or at least 1,000 m²/g. In other embodiments, the particles can have a BET surface area no greater than 100 m²/g, no greater than 75 m²/g, no greater than 50 m²/g, no greater than 25 m²/g, no greater than 10 m²/g, no greater than 5 m²/g, or no greater than 1 m²/g. In some embodiments, the particles can have a BET surface area between 1-100 m²/g, between 50-250 m²/g, between 250-500 m²/g, between 500-1,000 m²/g, or between 250-750 m²/g.

The particles can have an average particle size from 1-10,000 μm, from 100-500 μm, from 1-100 μm, from 1-50 μm, from 50-250 μm, from 250-500 μm, from 500-1,000 μm, from 1,000-5,000 μm, from 1,000-10,000 μm, or from 5,000-1,000 μm. In other embodiments, the particles can have an average particle size from 1-10,000 nm, from 1-5,000 nm, from 1-1,000 nm, from 1-500 nm, from 1-250 nm, from 1-100 nm, from 50-250 nm, from 250-750 nm, from 500-1,000 nm, from 750-1,500 nm, from 1,000-2,500 nm, from 2,500-7,500 nm, or from 5,000-10,000 nm.

In certain implementations, the wound care system can include an active agent, which may be a single therapeutic agent or a mixture of therapeutic agents. Exemplary active agents include antimicrobials, analgesics and anti-inflammatories, as well as combinations thereof. In certain implementations, the wound care system can include an antimicrobial agent, for example anti-bacterial agents, anti-mycobacterial agents, anti-viral agents, anti-fungal agents, and anti-parasite agents. Suitable antimicrobials include antibiotics, antimicrobial peptides and metallic compounds. In certain implementations, the wound care system include a silver antimicrobial agent, a QAS, or a combination thereof.

In some implementations, the active agent can include an antibiotic, for example, streptomycin, neomycin, kanamycin, amikacin, gentamycin, tobramycin, sisomicin, arbekacin, apramycin, netilmicin, paromomycin, spectinomycin, ciprofloxacin, levofloxacin, lomefloxacin, moxifloxacin, norfloxacin, ofloxacin, sparfloxacin, trvafloxacin, gatifloxacin, gemifloxacin, cinoxacin, nalidixic acid, azithromycin, clarithromycin, dirithromycin, erythromycin, roxithromycin, troleandomycin, telithromycin, spectinomycin, indolicidin, defensin, cecropin, magainin, vancomycin, teicoplanin, telavancin, ramoplanin, decaplanin, bleomycin, colistin (polymyxin E), colistin A (polymyxin E1), colistin B (polymyxin E2), colistin sulfate, colistimethate sodium, actinomycin, bacitracin, polymyxin B, gentamicin, gentamicin sulfate, neomycin, kanamycin, tobramycin, metronidazole, clotrimazole, secnidazole, ornidazole, tinidazole, linezolid, doxycycline, tetracycline, oxytetracycline, chlortetracycline, demeclocycline, lymecycline, meclocycline, methacycline, minocycline, rolitetracycline, and tigecycline.

In certain implementations, the active agent can include an analgesic, for example capsaicin, diclofenac, lidocaine, benzocaine, methyl salicylate, trolamine, prilocaine, pramoxine, dibucaine, phenol, tetracaine, camphor, dyclonine, or menthol.

In certain implementations, the agent includes an anti-inflammatory, for example alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lornoxicam, loteprednol etabonate, meclofenamate sodium, meclofenamic acid, meclorisone dibutyrate, mefenamic acid, mesalamine, meseclazone, methylprednisolone suleptanate, morniflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, and zomepirac sodium.

In some implementations, the active agent can include a cannabinoid or hemp-derived compound. In certain implementations the cannabinoid or hemp-derived compound can include tetrahydrocannabinol, cannabidiol, cannabinol, cannabichromene, tetrahydrocannabivarin, cannabidivarin, cannabigerol, or a combination thereof.

In some implementations, the active agent can include a growth factor, for example keratinocyte growth factor (KGF), platelet derived growth factor (PDGF), transforming growth factor-beta (TGFβ), interleukin, activin, colony stimulating factor, connective tissue growth factor (CTGF), epidermal growth factor (EGF), Epigen, erythropoietin, fibroblast growth factor (FGF), galectin, hepatoma-derived growth factor (HDGF), hepatocyte growth factor, insulin-like growth factor binding protein (IGFBP), insulin-like growth factor, insulin, leptin, macrophage migration inhibitory factor, melanoma inhibitory factor, myostatin, noggin, nephroblastoma overexpressed (NOV), omentin, oncostatinM, osteopontin, osteoprotogerin (OPG), periostin, placenta growth factor, placental lactogen, prolactin, RANK ligand, retinol binding protein, stem cell factor, transforming growth factor, and vascular endothelial growth factor (VEGF). This includes associated isoforms from these growth factor families.

### Additional Embodiments

1. A wound dressing comprising:
a hydrophilic foam layer having a proximal side and a distal side; and
an active layer comprising an active-layer antimicrobial agent, wherein the active layer is disposed on the proximal side of the foam layer.
2. The dressing of any preceding embodiment, wherein the active layer comprises a QAS,
preferably K21, optionally in combination with at least one additional therapeutic agent.
3. The dressing of any preceding embodiment, wherein the active layer is detachable from the foam layer.
4. The dressing of any preceding embodiment, wherein the active layer comprises a bioresorbable polymer.
5. The dressing of any preceding embodiment, wherein the active layer comprises a breathable and non-occlusive polymer fiber mat.
6. The dressing of any preceding embodiment, wherein the active layer comprises a fibrous polymer mat.
7. The dressing of any preceding embodiment, wherein the active layer comprises a fibrous bioresorbable polymer mat.
8. The dressing of any preceding embodiment, wherein the active layer comprises an electrospun polymer mat.
9. The dressing of any preceding embodiment, wherein the active layer comprises an electrospun bioresorbable polymer mat.
10. The dressing of any preceding embodiment, wherein the bioresorbable polymer comprises saccharides, surfactants (e.g., poloxamers, etc.), collagen or gelatin, alginate, cellulose-based or carboxymethyl cellulose (CMC), polyvinyl alcohol (PVA or PVOH), polyethylene oxide (PEO), polylactic acid (PLA), glycolic acid, polycaprolactone (PCL), a copolymer thereof, or a combination thereof.
11. The dressing of any preceding embodiment, wherein the bioresorbable polymer comprises PLGA, PLA, PVA, PEO, CMC, or a combination thereof.
12. The dressing of any preceding embodiment, wherein the bioresorbable polymer comprises PVA, PEO, CMC, or a combination thereof.
13. The dressing of any preceding embodiment, wherein the active layer comprises a bioresorbable polymer fibers having an average fiber diameter from 10-1,000 nm, from 10-100 nm, from 10-50 nm, from 50-100 nm, from 50-250 nm, from 75-500 nm, from 100-250 nm, from 250-500 nm, or from 500-1,000 nm.
14. The dressing of any preceding embodiment, wherein the active layer comprises a bioresorbable polymer having a basis weight from 0.5-200 gsm, from 0.5-25 gsm, from 5-25 gsm, from 25-50 gsm, from 50-100 gsm, from 50-150 gsm, or from 100-200 gsm.
15. The dressing of any preceding embodiment, wherein the active layer comprises a first active layer and a second active layer, the second active layer disposed between the first active layer and the foam layer.
16. The dressing of any preceding embodiment, wherein the first active layer comprises a first active layer antimicrobial agent.
17. The dressing of any preceding embodiment, wherein the second active layer comprises a second active layer antimicrobial agent.
18. The dressing of any preceding embodiment, wherein the first active layer comprises a QAS, preferably K21, optionally in combination with at least one additional first active layer therapeutic agent.
19. The dressing of any preceding embodiment, wherein the second active layer comprises a QAS, preferably K21, optionally in combination with at least one additional second active layer therapeutic agent.
20. The dressing of any preceding embodiment, wherein the hydrophilic foam comprises a foam-layer antimicrobial agent.
21. The dressing of any preceding embodiment, wherein the hydrophilic foam comprises a foam-layer antimicrobial agent dispersed throughout the foam layer.
22. The dressing of any preceding embodiment, wherein the hydrophilic foam comprises a foam-layer antimicrobial agent on the distal surface of the foam layer, the proximal surface of the foam layer, or on both the distal and proximal surfaces of the foam layer.
23. The dressing of any preceding embodiment, wherein the foam-layer comprises a QAS, preferably K21, optionally in combination with at least one additional foam-layer therapeutic agent.
24. The dressing of any preceding embodiment, wherein the foam-layer antimicrobial agent comprises a particulate QAS, preferably particulate K21.
25. The dressing of any preceding embodiment, further comprising a barrier layer disposed on the distal side of the foam layer.
26. The dressing of any preceding embodiment, further comprising a release liner disposed between the foam layer and the active layer.
27. The dressing of any preceding embodiment, wherein the additional active layer therapeutic agent comprises an antimicrobial agent, an analgesic, an anti-inflammatory, a cannabinoid, a growth factor, or a combination thereof.
28. The dressing of any preceding embodiment, wherein the additional first active layer therapeutic agent comprises an antimicrobial agent, an analgesic, an anti-inflammatory, a cannabinoid, a growth factor, or a combination thereof.
29. The dressing of any preceding embodiment, wherein the additional second active layer therapeutic agent comprises an antimicrobial agent, an analgesic, an anti-inflammatory, a cannabinoid, a growth factor, or a combination thereof.
30. The dressing of any preceding embodiment, wherein the additional foam layer therapeutic agent comprises an antimicrobial agent, an analgesic, an anti-inflammatory, a cannabinoid, a growth factor, or a combination thereof.
31. A method of treating a wound, comprising contacting the wound with the dressing of any preceding embodiment.
32. The method of any preceding embodiment, wherein the wound comprises a trauma wound, surgical wound, burn wound, ulcer wound, or a combination thereof.
33. A kit comprising the dressing of any preceding embodiment, and a liquid spray composition comprising a spray antimicrobial agent.
34. The kit of any preceding embodiment, wherein the liquid spray composition comprises a QAS, preferably K21, optionally in combination with at least one additional liquid spray therapeutic agent.
35. The kit of any preceding embodiment, wherein the additional liquid spray therapeutic agent comprises an antimicrobial agent, an analgesic, an anti-inflammatory, a cannabinoid, a growth factor, or a combination thereof.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

What is claimed is:

1. A wound dressing comprising:
a hydrophilic foam layer having a proximal side and a distal side; and
an active layer comprising an active-layer antimicrobial agent comprising a quaternary ammonium siloxane, wherein the active layer is disposed on the proximal side of the foam layer.

2. The dressing of claim 1, wherein the antimicrobial agent comprises K21, optionally in combination with at least one additional therapeutic agent.

3. The dressing of claim 1, wherein the active layer is detachable from the foam layer.

4. The dressing of claim 1, wherein the active layer comprises a bioresorbable polymer.

5. The dressing of claim 4, wherein the bioresorbable polymer comprises surfactants, collagen, gelatin, alginate, cellulose-based polymer, polyvinyl alcohol, polyethylene oxide, polylactic acid, glycolic acid, polycaprolactone, a copolymer thereof, or a combination thereof.

6. The dressing of claim 4, wherein the bioresorbable polymer comprises carboxymethyl cellulose, polyvinyl alcohol, polyethylene oxide or a combination thereof.

7. The dressing of claim 1, wherein the active layer comprises a bioresorbable polymer fibers having an average fiber diameter from 10-1,000 nm.

8. The dressing of claim 1, wherein the active layer comprises a bioresorbable polymer having a basis weight from 0.5-200 gsm.

9. The dressing of claim 1, wherein the active layer comprises a first active layer and a second active layer, the second active layer disposed between the first active layer and the foam layer.

10. The dressing of claim 9, wherein the first active layer comprises the quaternary ammonium siloxane.

11. The dressing of claim 9, wherein the second active layer comprises the quaternary ammonium siloxane.

12. The dressing of claim 1, wherein the hydrophilic foam comprises a foam-layer antimicrobial agent.

13. The dressing of claim 12, wherein the hydrophilic foam comprises a foam-layer antimicrobial agent on the distal surface of the foam layer, the proximal surface of the foam layer, or on both the distal and proximal surfaces of the foam layer.

14. The dressing of claim 12, wherein the foam-layer antimicrobial agent comprises a quaternary ammonium siloxane.

15. The dressing of claim 12, wherein the foam-layer antimicrobial agent comprises K21.

16. The dressing of claim 12, wherein the foam-layer antimicrobial agent comprises particulate K21.

17. The dressing of claim 1, further comprising a barrier layer disposed on the distal side of the foam layer.

18. The dressing of claim 1, further comprising a release liner disposed between the foam layer and the active layer.

19. A method of treating a wound, comprising contacting the wound with the dressing of claim 1.

20. A kit comprising the dressing of claim 1, and a liquid spray composition comprising a spray antimicrobial agent.
